(19) United States
(12) Patent Application Publication
(10) Pub. No.: US 2025/0391526 A1
(43) Pub. Date: Dec. 25, 2025

(54) TECHNOLOGIES FOR PATIENT DOCUMENTATION IN A HEALTHCARE FACILITY USING VOICE INPUTS

(71) Applicant: Hill-Rom Services, Inc., Batesville, IN (US)

(72) Inventors: Dan R. Tallent, Hope, IN (US); Eric D. Agdeppa, Cincinnati, OH (US); Kenneth L. Lilly, West Chester, OH (US); Catherine J. Harb, Neptune Beach, FL (US); Timothy J. Receveur, Guilford, IN (US); John S. Schroder, Apex, NC (US); Richard J. Schuman, Cary, NC (US)

(73) Assignee: Hill-Rom Services, Inc., Batesville, IN (US)

(21) Appl. No.: 19/304,650

(22) Filed: Aug. 20, 2025

Related U.S. Application Data
(63) Continuation of application No. 17/887,016, filed on Aug. 12, 2022.
(60) Provisional application No. 63/236,104, filed on Aug. 23, 2021.

(51) Int. Cl. G16H 10/60; G16H 40/20; G16H 40/40; G16H 40/67

(52) U.S. Cl. CPC G16H 10/60; G16H 40/20; G16H 40/40; G16H 40/67

(57) ABSTRACT

Technologies for efficiently producing documentation from voice data in a healthcare facility may include a compute device. The compute device may include circuitry configured to obtain, from a caregiver and in response to a determination that the caregiver is located in a room with a patient in a healthcare facility, voice data indicative of spoken information pertaining to the patient. The circuitry may be additionally configured to produce, from the obtained voice data, textual data indicative of the spoken information. Further, the circuitry may be configured to provide the textual data to another device for storage or presentation.

### CROSS-REFERENCE TO RELATED APPLICATION

This application is a continuation of U.S. application Ser. No. 17/887,016, filed Aug. 12, 2022, which claimed the benefit, under 35 U.S.C. § 119 (e), of U.S. Provisional Patent Application No. 63/236,104, filed Aug. 23, 2021, each of which is hereby expressly incorporated by reference herein in its entirety.

### BACKGROUND

The present disclosure relates to producing documentation relating to healthcare services and more particularly to efficiently producing documentation from information spoken by caregivers in a healthcare facility.

In a typical healthcare facility (e.g., a hospital), caregivers (e.g., nurses, doctors, etc.) provide services under a variety of pressures, including the need to provide prompt and timely care to many patients during a limited time frame, and the need to provide customized care that takes into account information that was developed about a given patient, such as from previous visits to the patient's room (e.g., on hospital rounds) or medical procedures (e.g., surgery) that may have been performed on the patient. However, given the fast paced nature of providing healthcare services in a healthcare facility, it is difficult for a caregiver to fully document information that was learned about a patient during a recent interaction with the patient, before moving on to another patient.

In the context of an operating room, a team of caregivers, such as surgeons, nurses, and anesthesiologists cooperate in a carefully coordinated manner to perform a complex medical procedure that may involve many pre- and post-operative steps and the use of high-tech medical equipment. As such, the full attention of the caregivers is focused on performing the procedure. Accordingly, information that may have been developed during the course of the procedure, such as observations about the condition of the patient during the procedure, information about the settings of medical equipment used at different stages in the procedure, and actions that should be performed post-operatively, may not be accurately or completely retained by the caregivers after the operation is complete (i.e., when the information would typically be documented in an operation note). As a consequence, when another caregiver subsequently tends to a patient, that caregiver may not have access to all of the information that was developed from a previous interaction with the patient, as some portion of the information may not have been documented.

### SUMMARY

The present application discloses one or more of the features recited in the appended claims and/or the following features which, alone or in any combination, may comprise patentable subject matter:

According to an aspect of the present disclosure, a compute device may include circuitry configured to obtain, from a caregiver, voice data indicative of spoken information pertaining to a patient. The compute device may obtain the voice data in response to a determination that the caregiver is located in a room with a patient in a healthcare facility (e.g., based on information from a real time location tracking system). The circuitry may additionally be configured to produce, from the obtained voice data, textual data indicative of the spoken information. Further, the circuitry may be configured to provide the textual data to another device for storage or presentation. The caregiver may be associated with a first shift and, in some embodiments, the circuitry may be configured to determine that a change from a first shift to a second shift has occurred, determine that a second caregiver associated with a second shift is assigned to the patient, and provide, to the second caregiver and in response to the determination that the shift change has occurred, a notification of the textual data. The circuitry, in some embodiments, may be configured to determine that a second caregiver has entered a room associated with the patient, and provide, to the second caregiver and in response to the determination that the second caregiver has entered the room, a notification of the textual data.

In some embodiments, the circuitry of the compute device may be configured such that providing the notification includes providing the notification to a mobile compute device carried by the second caregiver. The circuitry, in some embodiments, may be configured to prompt the second caregiver to acknowledge that the textual data has been reviewed. Additionally or alternatively, the circuitry of the compute device may be configured to determine whether the second caregiver has reviewed the textual data within a predefined time period and provide, in response to a determination that the second caregiver has not reviewed the textual data within the predefined time period, a reminder to the second caregiver to review the textual data. In some embodiments, the circuitry of the compute device may be configured to determine an identity of the patient based on patient designation data provided by the caregiver or based on a determination that the caregiver is located in a room assigned to the patient. Additionally or alternatively, the circuitry may be configured to provide the textual data to a bedside display device.

In some embodiments, the circuitry may be configured to display the textual data to the caregiver for review and editing before the textual data is provided to another device for storage or presentation. The caregiver may be one of multiple caregivers in an operating room in which a medical procedure is performed on the patient and the circuitry may be further configured to determine, from the voice data, an identity of the caregiver that provided the spoken information from among the plurality of the caregivers in the operating room.

The circuitry of the compute device, in some embodiments, may be configured to produce the textual data using a machine learning model trained to convert speech to text. Additionally or alternatively, the circuitry of the compute device may be configured to correct one or more words in the textual data based on a context in which the one or more words were spoken. Further, the circuitry may be configured such that to correct one or more words based on a context in which the one or more words were spoken comprises to correct one or more words based on data indicative of a medical procedure being performed when the one or more words were spoken, a status of the patient when the one or more words were spoken, a determined location of the speaker, words previously spoken by the speaker, or one or more predefined words associated with predefined commands. In some embodiments, the circuitry may be configured to supplement the textual data with tag data indicative of a context of the textual data. The circuitry may also be configured such that to supplement the textual data with tag data includes supplementing the textual data with time stamp data indicative of times at which the spoken information was obtained. In some embodiments, the circuitry may be configured such that supplementing the textual data with tag data includes supplementing the textual data with caregiver identification data indicative of an identity of a speaker of the spoken information.

In some embodiments, the circuitry may be configured such that supplementing the textual data with tag data includes supplementing the textual data with speaker location data indicative of a location of a speaking caregiver associated with the spoken information. Additionally or alternatively, the circuitry may be configured such that supplementing the textual data with tag data includes supplementing the textual data with speaker direction data indicative of a direction a speaking caregiver was facing when the spoken information was obtained. In some embodiments, the circuitry may be configured such that supplementing the textual data with tag data includes supplementing the textual data with a listing of equipment located in the operating room in which the medical procedure is performed on the patient. Additionally or alternatively, the circuitry of the compute device may be configured such that supplementing the textual data with tag data includes supplementing the textual data with data indicative of a type of medical procedure performed on the patient.

In some embodiments, the circuitry of the compute device may be configured such that supplementing the textual data with tag data includes supplementing the textual data with procedure stage data that may be indicative of a present stage of the medical procedure performed on the patient when the spoken information was obtained. The circuitry of the compute device, in some embodiments, may be configured such that supplementing the textual data with tag data includes supplementing the textual data with patient status data indicative of a status of the patient when the spoken information was obtained. Additionally or alternatively, the circuitry may be configured such that supplementing the textual data with tag data includes supplementing the textual data with equipment status data indicative of a status of equipment present in the room in which the medical procedure is performed.

In some embodiments, supplementing the textual data with tag data includes supplementing the textual data with tag data indicative of an incision site, an incision type, a location or diagram of incisions relative to each other, a size of a laparoscopic port used, an intra-operative finding, an identification of a pathology, stages of the medical procedure carried out from first incision to closure, ligation of one or more vessels, identification of an implant or prosthesis used in the medical procedure, excised tissue, anatomy notably identified, closure time, one or more materials used for closure, one or more intraoperative complications noted, one or more specimens obtained, blood loss, or one or more actions to be taken post-operatively. The circuitry may be additionally or alternatively configured to supplement the textual data with signature data that may be indicative of a signature and date associated with a caregiver who spoke the spoken information represented in the textual data.

In some embodiments, the circuitry of the compute device may be configured to provide the tag data to the other device for storage or presentation. The compute device, in some embodiments, may be part of a medical device used in the medical procedure on the patient. The circuitry may be configured such that providing the textual data to another device includes providing the textual data to at least one of an electronic medical records system, a device in a patient room, a device in an operating room, a personal computer, a device operating a web browser, a mobile device, an augmented reality presentation device, a projection device, or a wearable device. The circuitry may, in some embodiments, be configured to reduce ambient noise in the voice data.

In another aspect of the present disclosure, a method may include obtaining, by a compute device and from a caregiver, voice data indicative of spoken information pertaining to a patient, in response to a determination that the caregiver is located in a room with the patient in a healthcare facility (e.g., based on information from a real time location tracking system). The method may additionally include producing, by the compute device and from the obtained voice data, textual data indicative of the spoken information. Further, the method may include providing, by the compute device, the textual data to another device for storage or presentation. In some embodiments, the caregiver may be associated with a first shift and the method may further include determining, by the compute device, that a change from a first shift to a second shift has occurred, determining, by the compute device, that a second caregiver associated with a second shift is assigned to the patient, and providing, by the compute device, to the second caregiver and in response to the determination that the shift change has occurred, a notification of the textual data.

The method, in some embodiments, may additionally include determining, by the compute device, that a second caregiver has entered a room associated with the patient. Further, the method may include providing, by the compute device, to the second caregiver and in response to the determination that the second caregiver has entered the room, a notification of the textual data. The method may include providing the notification to a mobile compute device carried by the second caregiver. In some embodiments, the method includes prompting the second caregiver to acknowledge that the textual data has been reviewed. In some embodiments, the method includes determining, by the compute device, whether the second caregiver has reviewed the textual data within a predefined time period and providing, by the compute device and in response to a determination that the second caregiver has not reviewed the textual data within the predefined time period, a reminder to the second caregiver to review the textual data.

In some embodiments, the method includes determining, by the compute device, an identity of the patient based on patient designation data provided by the caregiver or based on a determination that the caregiver is located in a room assigned to the patient. The method may include providing, by the compute device, the textual data to a bedside display device. Additionally or alternatively, the method may include displaying, by the compute device, the textual data to the caregiver for review and editing before the textual data is provided to another device for storage or presentation. In some embodiments, the caregiver is one of multiple caregivers in an operating room in which a medical procedure is performed on the patient and the method may further include determining, by the compute device and from the voice data, an identity of the caregiver that provided the spoken information from among the plurality of the caregivers in the operating room. In some embodiments, the method additionally includes producing, by the compute device, the textual data using a machine learning model trained to convert speech to text.

The method may further include correcting, by the compute device, one or more words in the textual data based on a context in which the one or more words were spoken. Correcting one or more words based on a context in which the one or more words were spoken may include correcting one or more words based on data indicative of a medical procedure being performed when the one or more words were spoken, a status of the patient when the one or more words were spoken, a determined location of the speaker, words previously spoken by the speaker, or one or more predefined words associated with predefined commands. In some embodiments, the method includes supplementing the textual data with tag data indicative of a context of the textual data. Supplementing the textual data with tag data may include supplementing the textual data with time stamp data indicative of times at which the spoken information was obtained. Additionally or alternatively, supplementing the textual data with tag data may include supplementing the textual data with caregiver identification data indicative of an identity of a speaker of the spoken information.

In some embodiments, supplementing the textual data with tag data includes supplementing the textual data with speaker location data indicative of a location of a speaking caregiver associated with the spoken information, supplementing the textual data with speaker direction data indicative of a direction a speaking caregiver was facing when the spoken information was obtained, supplementing the textual data with a listing of equipment located in the operating room in which the medical procedure is performed on the patient, supplementing the textual data with data indicative of a type of medical procedure performed on the patient, and/or supplementing the textual data with procedure stage data indicative of a present stage of the medical procedure performed on the patient when the spoken information was obtained.

Supplementing the textual data with tag data, in some embodiments, may include supplementing the textual data with patient status data indicative of a status of the patient when the spoken information was obtained and/or supplementing the textual data with equipment status data indicative of a status of equipment present in the room in which the medical procedure is performed. In some embodiments, supplementing the textual data with tag data includes supplementing the textual data with tag data indicative of an incision site, an incision type, a location or diagram of incisions relative to each other, a size of a laparoscopic port used, an intra-operative finding, an identification of a pathology, stages of the medical procedure carried out from first incision to closure, ligation of one or more vessels, identification of an implant or prosthesis used in the medical procedure, excised tissue, anatomy notably identified, closure time, one or more materials used for closure, one or more intraoperative complications noted, one or more specimens obtained, blood loss, or one or more actions to be taken post-operatively.

The method may additionally or alternatively include supplementing, by the compute device, the textual data with signature data indicative of a signature and date associated with a caregiver who spoke the spoken information represented in the textual data. In some embodiments, the method includes providing, by the compute device, the tag data to the other device for storage or presentation. Providing the textual data to another device may include providing the textual data to at least one of an electronic medical records system, a device in a patient room, a device in an operating room, a personal computer, a device operating a web browser, a mobile device, an augmented reality presentation device, a projection device, or a wearable device. In some embodiments, the method includes reducing, by the compute device, ambient noise in the voice data.

In another aspect of the present disclosure, one or more machine-readable storage media may include instructions stored thereon. In response to being executed, the instructions may cause a compute device to obtain, from a caregiver, voice data indicative of spoken information pertaining to a patient. The instructions may cause the compute device to obtain the voice data in response to a determination that the caregiver is located in a room with a patient in a healthcare facility (e.g., based on information from a real time location tracking system). The instructions may further cause the compute device to produce, from the obtained voice data, textual data indicative of the spoken information. Additionally, the instructions may cause the compute device to provide the textual data to another device for storage or presentation. The caregiver may be associated with a first shift and in some embodiments, the instructions may cause the compute device to determine that a change from a first shift to a second shift has occurred, determine that a second caregiver associated with a second shift is assigned to the patient, and provide, to the second caregiver and in response to the determination that the shift change has occurred, a notification of the textual data.

The instructions may, in some embodiments, cause the compute device to determine that a second caregiver has entered a room associated with the patient and provide, to the second caregiver and in response to the determination that the second caregiver has entered the room, a notification of the textual data. In some embodiments, providing the notification includes providing the notification to a mobile compute device carried by the second caregiver. The one or more instructions may also cause the compute device to prompt the second caregiver to acknowledge that the textual data has been reviewed. In some embodiments, the one or more instructions may cause the compute device to determine whether the second caregiver has reviewed the textual data within a predefined time period and provide, in response to a determination that the second caregiver has not reviewed the textual data within the predefined time period, a reminder to the second caregiver to review the textual data.

The one or more instructions may, in some embodiments, cause the compute device to determine an identity of the patient based on patient designation data provided by the caregiver or based on a determination that the caregiver is located in a room assigned to the patient. The one or more instructions may cause the compute device to provide the textual data to a bedside display device. The instructions may, in some embodiments, cause the compute device to display the textual data to the caregiver for review and editing before the textual data is provided to another device for storage or presentation. The caregiver may be one of multiple caregivers in an operating room in which a medical procedure is performed on the patient and the one or more instructions may additionally cause the compute device to determine, from the voice data, an identity of the caregiver that provided the spoken information from among the caregivers in the operating room.

In some embodiments, the one or more instructions additionally cause the compute device to produce the textual data using a machine learning model trained to convert speech to text. The one or more machine-readable storage media may additionally cause the compute device to correct one or more words in the textual data based on a context in which the one or more words were spoken. In correcting one or more words based on a context in which the one or more words were spoken, the instructions may cause the compute device to correct one or more words based on data indicative of a medical procedure being performed when the one or more words were spoken, a status of the patient when the one or more words were spoken, a determined location of the speaker, words previously spoken by the speaker, or one or more predefined words associated with predefined commands. The instructions may additionally or alternatively cause the compute device to supplement the textual data with tag data indicative of a context of the textual data.

In supplementing the textual data with tag data, the instructions may cause the compute device to supplement the textual data with time stamp data indicative of times at which the spoken information was obtained, supplement the textual data with caregiver identification data indicative of an identity of a speaker of the spoken information, supplement the textual data with speaker location data indicative of a location of a speaking caregiver associated with the spoken information, supplement the textual data with speaker direction data indicative of a direction a speaking caregiver was facing when the spoken information was obtained, and/or supplement the textual data with a listing of equipment located in the operating room in which the medical procedure is performed on the patient.

In some embodiments, in supplementing the textual data with tag data, the instructions may cause the compute device to supplement the textual data with data indicative of a type of medical procedure performed on the patient, supplement the textual data with procedure stage data indicative of a present stage of the medical procedure performed on the patient when the spoken information was obtained, supplement the textual data with patient status data indicative of a status of the patient when the spoken information was obtained, and/or supplement the textual data with equipment status data indicative of a status of equipment present in the room in which the medical procedure is performed. Additionally or alternatively, the instructions may cause the compute device to supplement the textual data with tag data indicative of an incision site, an incision type, a location or diagram of incisions relative to each other, a size of a laparoscopic port used, an intra-operative finding, an identification of a pathology, stages of the medical procedure carried out from first incision to closure, ligation of one or more vessels, identification of an implant or prosthesis used in the medical procedure, excised tissue, anatomy notably identified, closure time, one or more materials used for closure, one or more intraoperative complications noted, one or more specimens obtained, blood loss, or one or more actions to be taken post-operatively.

In some embodiments, the one or more instructions may cause the compute device to supplement the textual data with signature data indicative of a signature and date associated with a caregiver who spoke the spoken information represented in the textual data. The one or more machine-readable storage media may also have instructions embodied thereon that cause the compute device to provide the tag data to the other device for storage or presentation. In some embodiments, the instructions may cause the compute device to provide the textual data to at least one of an electronic medical records system, a device in a patient room, a device in an operating room, a personal computer, a device operating a web browser, a mobile device, an augmented reality presentation device, a projection device, or a wearable device. The instructions may also cause the compute device to reduce ambient noise in the voice data.

Additional features, which alone or in combination with any other feature(s), such as those listed above and/or those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a diagram of a system for efficiently producing documentation from voice data in a healthcare facility;

FIG. 2 is a diagram of components of a compute device included in the system of FIG. 1;

FIGS. 3-8 are diagrams of at least one embodiment of a method for efficiently producing documentation from voice data in a healthcare facility that may be performed by the system of FIG. 1;

FIG. 9 is a diagram of a flow of data through multiple functional components as the method of FIGS. 3-8 is performed; and

FIGS. 10-13 are flow diagrams of the production of documentation from voice data and the distribution of the documentation to caregivers in a healthcare facility.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one of A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

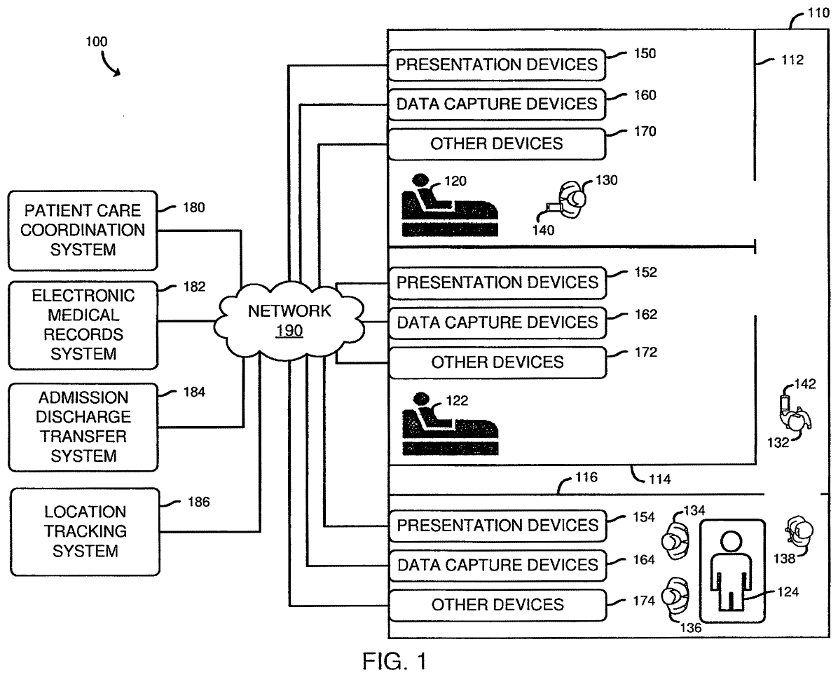

Referring now to FIG. 1, a system 100 for efficiently producing documentation from voice data in a healthcare facility 110 includes a healthcare facility 110 (e.g., a hospital) with multiple rooms 112, 114, 116 in which caregivers 130, 132, 134, 136, 138, provide care to patients 120, 122, 124. In the illustrative embodiments, at least some of the caregivers (e.g., 130, 132) carry mobile compute devices 140, 142 (e.g., smartphones, tablets, etc.) configured to provide information to the corresponding caregiver regarding patients in the healthcare facility 110, such as rounding notes (e.g., notes regarding the status of a patient observed by a caregiver who visited the patient's room (e.g., room 112, 114) during a round), notes regarding a medical procedure, such as a surgery, performed on a patient (e.g., the patient 124), and/or other information. Further, the mobile compute devices 140, 142 in the illustrative embodiment, are configured to receive information from the corresponding caregivers, such as rounding notes and/or medical procedure notes, and provide the information to one or more other compute devices (also referred to herein as “devices”) 140, 142, 150, 152, 154, 160, 162, 164, 170, 172, 174, 180, 182, 184, 186 in the system 100.

As shown in FIG. 1, each room 112, 114, 116 in the illustrative healthcare facility 110 includes presentation devices 150, 152, 154, data capture devices 160, 162, 164, and other devices 170, 172, 174. The presentation devices 150, 152, 154 may each be embodied as any device or circuitry configured to present information to a person (e.g., a caregiver) visually and/or audibly. For example, one or more of the presentation devices 150, 152, 154 may be embodied as a display device (e.g., an interactive whiteboard, a human-machine interface (HMI), etc.) that is free-standing or mounted to a wall, patient bed, other patient support apparatus, or other device in the room. Each data capture device 160, 162, 164 may be embodied as any device or circuitry configured to obtain data from the environment, such as audio data (e.g., through one or more microphones), visual data (e.g., through one or more cameras), user-entered data (e.g., typed information, selection(s) of displayed data, such as on a touch screen, etc.), and/or other data. The other devices 170, 172, 174 may be embodied as medical equipment, such as patient support apparatuses (e.g., beds, chairs, etc.), patient status monitoring devices (e.g., pulse oximeter device(s), electrocardiogram devices, etc.), surgical instruments, nurse call devices, networking devices (e.g., hubs, switches, routers, gateways, etc.), and/or other electronic or electromechanical devices in the corresponding room 112, 114, 116. While shown separately, in some embodiments, two or more of the devices 150, 160, 170 in the room 112 may be combined into a single device (e.g., in a single housing, configured to operate together, etc.). Likewise, two or more of the devices 152, 162, 172 in the room 114 may be combined, and two or more of the devices 154, 164, 174 in the room 116 may be combined.

The patient care coordination system 180 may be embodied as any device(s) (e.g., one or more server compute devices) located on premises or remotely from the healthcare facility 110 (e.g., in a cloud data center) configured to enable communication among the caregivers at the healthcare facility 110, receive information from device(s) at the healthcare facility 110, and notify corresponding caregivers (e.g., caregivers assigned to a team associated with a particular patient to whom the information pertains) of the information. The electronic medical records (EMR) system 182 may be embodied as any device(s) (e.g., one or more server compute devices) located on premises or remotely from the healthcare facility 110 (e.g., in a cloud data center) configured to obtain electronic (e.g., digital) medical record data pertaining to patients, store the electronic medical record data (e.g., in one or more data storage devices), and provide the electronic medical record data (e.g., upon request) to an authenticated compute device (e.g., to a mobile compute device 140, 142) of a caregiver (e.g., a caregiver 130, 132).

Still referring to FIG. 1, the admission, discharge, transfer (ADT) system 184 may be embodied as any device(s) (e.g., one or more server compute devices) located on premises or remotely from the healthcare facility 110 (e.g., in a cloud data center) configured to store data indicative of patients that have been admitted to the healthcare facility, such as when the patients were admitted, unique identifiers associated with the patients, references to medical record data (e.g., located in the EMR system 182) associated with each patient, data indicative of which patients have been discharged from the healthcare facility 110 and when, and data indicative of a room assigned to each patient. The location tracking system 186 may be embodied as any device(s) configured to track the locations of people and devices (e.g., medical equipment, patient support apparatuses, etc.) throughout the healthcare facility 110. In some embodiments, the location tracking system 186 may utilize data captured by the data capture devices 160, 162, 164 to determine the locations of people and/or equipment in the healthcare facility (e.g., using facial recognition, object recognition, voice recognition, etc.) to identify the corresponding people and/or equipment. In some embodiments, caregivers and/or equipment may have tracking tags attached thereto (e.g., attached to their clothing, affixed to the equipment, etc.) that are detectable by corresponding devices (e.g., near field communication (NFC) devices, bar code readers, etc.) that report detections of the tracking tags to the compute device(s) (e.g., server compute device(s)) of the location tracking system 186.

In operation, the system 100, using one or more of the compute devices 140, 142, 150, 152, 154, 160, 162, 164, 170, 172, 174, 180, 182, 184, 186 described above, obtains voice data from one or more caregivers and converts the voice data into textual data to be stored and/or presented on an as-needed or as-requested basis. As such, the system 100 frees up the caregivers from the time-consuming task of manually entering textual notes pertaining to a patient during hospital rounds or in association with a medical procedure (e.g., surgical operation) performed on the patient. Furthermore, and as described in more detail herein, the system 100 may supplement the textual data with metadata (e.g., also referred to herein as tag data) indicative of contextual information associated with the textual data, such as identifiers of the caregivers who provided certain information (e.g., caregivers who spoke the information that has been converted to text), when the information was spoken, the patient to whom the information pertains, the location of the speaker of the information when the information was spoken, the stage of a medical procedure associated with a medical procedure during which the information was spoken, the settings of one or more devices (e.g., medical devices) at the time the information was spoken, diagrams and/or other visual information (e.g., locations of incisions made during a surgery, etc.). As such, the system 100 provides a more complete record, with significantly greater efficiency, than conventional systems in which caregivers are relied on to recall and manually enter information pertaining to patients in the course of performing hospital rounds and/or during the course of performing surgeries or other medical procedures on patients. Moreover, and as described in the more detail herein, the system 100 may determine to provide pertinent information to caregivers without their express request to do so (e.g., upon a change in care teams assigned to one or more patients, upon detecting that a caregiver has entered a room associated with a patient), to increase the likelihood that caregivers are equipped with pertinent information that could improve the care they provide to patients.

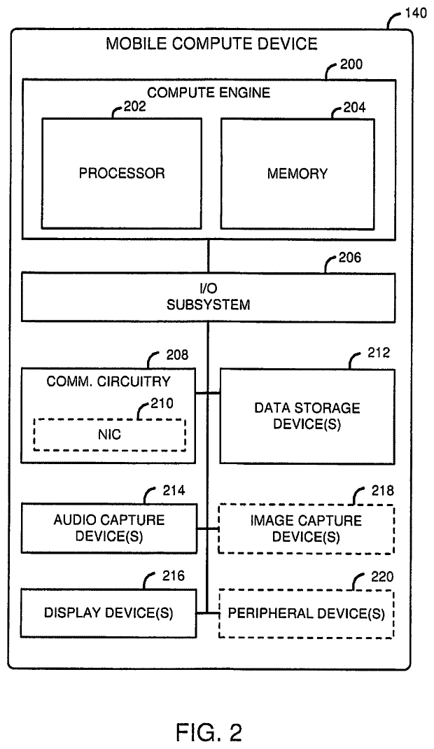

Referring now to FIG. 2, the illustrative mobile compute device 140 includes a compute engine 200, an input/output (I/O) subsystem 206, communication circuitry 208, one or more data storage devices 212, one or more audio capture devices 214, and one or more display devices 216. In some embodiments, the mobile compute device 140 may additionally include one or more image capture devices 218 and/or one or more peripheral device(s) 220. Additionally, in some embodiments, one or more of the illustrative components may be incorporated in, or otherwise form a portion of, another component.

The compute engine 200 may be embodied as any type of device or collection of devices capable of performing various compute functions described below. In some embodiments, the compute engine 200 may be embodied as a single device such as an integrated circuit, an embedded system, a field-programmable gate array (FPGA), a system-on-a-chip (SOC), or other integrated system or device. Additionally, in the illustrative embodiment, the compute engine 200 includes or is embodied as a processor 202 and a memory 204. The processor 202 may be embodied as any type of processor capable of performing the functions described herein. For example, the processor 202 may be embodied as a single or multi-core processor(s), a microcontroller, or other processor or processing/controlling circuit. In some embodiments, the processor 202 may be embodied as, include, or be coupled to an FPGA, an application specific integrated circuit (ASIC), reconfigurable hardware or hardware circuitry, or other specialized hardware to facilitate performance of the functions described herein.

The main memory 204 may be embodied as any type of volatile (e.g., dynamic random access memory (DRAM), etc.) or non-volatile memory or data storage capable of performing the functions described herein. Volatile memory may be a storage medium that requires power to maintain the state of data stored by the medium. In some embodiments, all or a portion of the main memory 204 may be integrated into the processor 202. In operation, the main memory 204 may store various software and data used during operation such as voice data, textual data produced from the voice data, tag data indicative of contextual information associated with the textual data, patient medical record data, applications, libraries, and drivers.

The compute engine 200 is communicatively coupled to other components of the mobile compute device 140 via the I/O subsystem 206, which may be embodied as circuitry and/or components to facilitate input/output operations with the compute engine 200 (e.g., with the processor 202 and the main memory 204) and other components of the mobile compute device 140. For example, the I/O subsystem 206 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O subsystem 206 may form a portion of a system-on-a-chip (SoC) and be incorporated, along with one or more of the processor 202, the main memory 204, and other components of the mobile compute device 140, into the compute engine 200.

The communication circuitry 208 may be embodied as any communication circuit, device, or collection thereof, capable of enabling communications over a network between the mobile compute device 140 and another device 142, 150, 152, 154, 160, 162, 164, 170, 172, 174, 180, 182, 184, 186. The communication circuitry 208 may be configured to use any one or more communication technology (e.g., wired or wireless communications) and associated protocols (e.g., Wi-Fi®, WiMAX, Bluetooth®, cellular, Ethernet, etc.) to effect such communication.

The illustrative communication circuitry 208 includes a network interface controller (NIC) 210. The NIC 210 may be embodied as one or more add-in-boards, daughter cards, network interface cards, controller chips, chipsets, or other devices that may be used by the mobile compute device 140 to connect with another device 142, 150, 152, 154, 160, 162, 164, 170, 172, 174, 180, 182, 184, 186. In some embodiments, the NIC 210 may be embodied as part of a system-on-a-chip (SoC) that includes one or more processors, or included on a multichip package that also contains one or more processors. In some embodiments, the NIC 210 may include a local processor (not shown) and/or a local memory (not shown) that are both local to the NIC 210. In such embodiments, the local processor of the NIC 210 may be capable of performing one or more of the functions of the compute engine 200 described herein. Additionally or alternatively, in such embodiments, the local memory of the NIC 210 may be integrated into one or more components of the mobile compute device 140 at the board level, socket level, chip level, and/or other levels.

Each data storage device 212 may be embodied as any type of device configured for short-term or long-term storage of data such as, for example, memory devices and circuits, memory cards, hard disk drives, solid-state drives, or other data storage device. Each data storage device 212 may include a system partition that stores data and firmware code for the data storage device 212 and one or more operating system partitions that store data files and executables for operating systems. Each audio capture device 214 may be embodied as any device or circuitry (e.g., a microphone) configured to obtain audio data (e.g., human speech) and convert the audio data to digital form (e.g., to be written to the memory 204 and/or one or more data storage devices 212). Each display device 216 may be embodied as any device or circuitry (e.g., a liquid crystal display (LCD), a light emitting diode (LED) display, a cathode ray tube (CRT) display, etc.) configured to display visual information (e.g., text, graphics, etc.) to a viewer (e.g., a caregiver or other user of the mobile compute device 140). Each image capture device 218 may be embodied as any device or circuitry (e.g., a camera) configured to obtain visual data from the environment and convert the visual data to digital form (e.g., to be written to the memory 204 and/or one or more data storage devices 212). Each peripheral device 220 may be embodied as any device or circuitry commonly found on a compute device, such as a keyboard, a mouse, or a speaker to supplement the functionality of the other components described above.

The compute devices 142, 150, 152, 154, 160, 162, 164, 170, 172, 174, 180, 182, 184, 186 may have components similar to those described in FIG. 2 with reference to the mobile compute device 140. The description of those components of the mobile compute device 140 is equally applicable to the description of components of the compute devices 142, 150, 152, 154, 160, 162, 164, 170, 172, 174, 180, 182, 184, 186. Further, it should be appreciated that any of the compute devices 140, 142, 150, 152, 154, 160, 162, 164, 170, 172, 174, 180, 182, 184, 186 may include other components, sub-components, and devices commonly found in computing devices, which are not discussed above in reference to the mobile compute device 140 and not discussed herein for clarity of the description. Further, while shown separately in FIG. 1, it should be understood that in some embodiments, one or more of the compute devices 140, 142, 150, 152, 154, 160, 162, 164, 170, 172, 174, 180, 182, 184, 186 may be combined or integrated into a single device (e.g., compute device). Additionally, while the components of a compute device may be shown as being housed in a single unit (e.g., housing), it should be understood that the components may be distributed across any distance and/or may be embodied as virtualized components (e.g., using one or more virtual machines utilizing hardware resources located in one or more data centers).

In the illustrative embodiment, the compute devices 140, 142, 150, 152, 154, 160, 162, 164, 170, 172, 174, 180, 182, 184, 186 are in communication via a network 190, which may be embodied as any type of wired or wireless communication network, including local area networks (LANs) or wide area networks (WANs), digital subscriber line (DSL) networks, cable networks (e.g., coaxial networks, fiber networks, etc.), cellular networks (e.g., Global System for Mobile Communications (GSM), Long Term Evolution (LTE), Worldwide Interoperability for Microwave Access (WiMAX), 3G, 4G, 5G, etc.), radio area networks (RAN), global networks (e.g., the internet), or any combination thereof, including gateways between various networks.

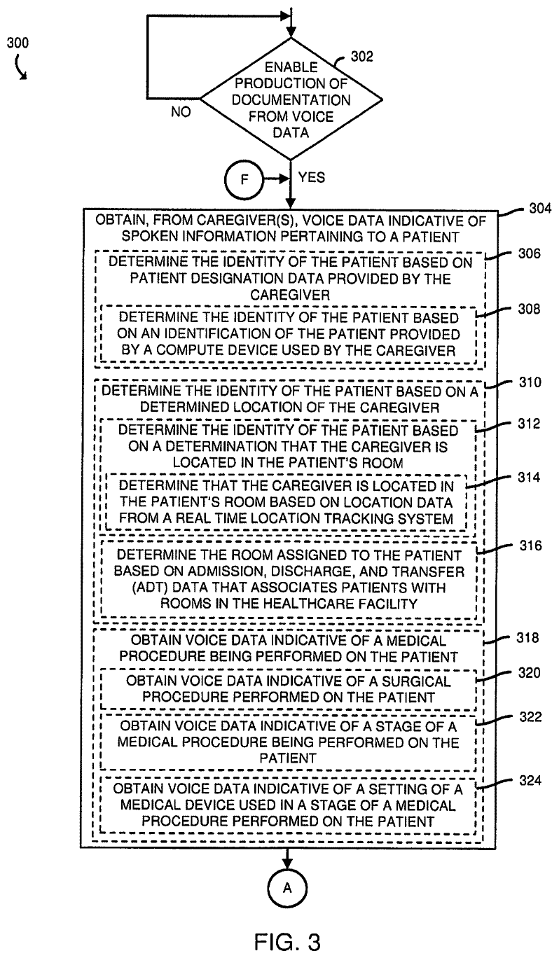

Referring now to FIG. 3, the system 100 (e.g., any compute device or combination of the compute devices operating cooperatively in the system 100, such as a mobile compute device 140, 142, and/or any one or more of the compute devices 150, 152, 154, 160, 162, 164, 170, 172, 174, 180, 182, 184, 186) may perform a method 300 for efficiently producing documentation from voice data in a healthcare facility (e.g., the hospital 110). The method 300, in the illustrative embodiment, begins with block 302, in which the system 100 determines whether to enable production of documentation from voice data. The system 100 may determine to enable production of documentation from voice data in response to a determination that a configuration setting (e.g., in a configuration file stored in a data storage device 212 and/or in memory 204) indicates to enable production of documentation from voice data, in response to a request (e.g., from a compute device) to enable production of documentation from voice data, in response to detection of a spoken request (e.g., spoken by a caregiver 130, 132, 134, 136, 138 and captured by an audio capture device 214) to produce documentation from voice data, and/or based other factors. Regardless, in response to a determination to enable production of documentation from voice data, the method 300 advances to block 304 in which the system 100 (e.g., the mobile compute device 140) obtains, from one or more caregivers (e.g., the caregiver 130), voice data indicative of spoken information pertaining to a patient (e.g., the patient 120). As an example, the mobile compute device 140 may capture (e.g., sample, record, etc.) one or more words spoken by the caregiver 130 using the audio capture device 214 (e.g., a microphone). In the illustrative embodiment, the system 100 may obtain the voice data in response to a determination that the caregiver (e.g., the caregiver 130) is presently located in the same room (e.g., the room 112) as the patient (e.g., the patient 120). For example, the system 100 may determine that the caregiver has entered the patient's room based on information from the real time location tracking system 186 (e.g., indicating the caregiver's present location) and information from the ADT system 184 (e.g., identifying the room assigned to the patient), and capture the caregiver's speech in response to a determination that the caregiver is located in the patient's room. Additionally or alternatively, the system 100 may determine that the caregiver is located in the patient's room based on other information (e.g., information provided by the caregiver (e.g., through the caregiver's mobile compute device 140) affirmatively indicating that the caregiver has entered the patient's room).

As indicated in block 306, the system 100 may determine the identity of the patient (e.g., the patient 120) based on patient designation data (e.g., the patient's name, a room number of the patient, an identification number of the patient, etc.) provided by the caregiver (e.g., the caregiver 130). In doing so, and as indicated in block 308, the system 100 may determine the identity of the patient based on an identification of the patient provided by a compute device (e.g., the mobile compute device 140) used by the caregiver (e.g., the caregiver 130). For example, the mobile compute device 140 may receive the patient designation data through selection, by the caregiver 130, of the patient's name on a touch screen of the mobile compute device 140 or may obtain the patient designation data through the audio capture device 214 if the caregiver 130 speaks the patient's name.

In some embodiments, the system 100 may determine the identity of the patient based on a determined location of the caregiver (e.g., the caregiver 130), as indicated in block 310. For example, and as indicated in block 312, the system 100 may determine the identity of the patient based on a determination that the caregiver (e.g., the caregiver 130) is located in the patient's room (e.g., the room 112). The system 100 may determine that the caregiver is located in the patient's room (e.g., the room 112) based on location data obtained from a real time location tracking system (e.g., the RLTS system 186), as indicated in block 314. That is, the location data may indicate, for example, that a location tracking badge (e.g., an NFC tag) worn by the caregiver 130 has been detected in the room 112, where the patient 120 is located. As indicated in the block 316, in making the determination of the identity of the patient, the system 100 may determine the room assigned to the patient based on admission, discharge, and transfer (ADT) data that associates patients with rooms in the healthcare facility 110. The ADT data may be provided by the ADT system 184 described with reference to FIG. 1.

In some embodiments, the method 300 may include obtaining voice data indicative of a medical procedure being performed on a patient, as indicated in block 318. For example, in the room 116 of FIG. 1, the caregivers 134, 136, 138 may perform a medical procedure on the patient 124. One or more data capture devices 164 in the room 116 may obtain voice data (e.g., spoken word(s)) from one or more the caregivers present in the room 116 and the spoken word(s) may indicate the type of medical procedure to be performed on the patient 124. As indicated in block 320, the system 100 may obtain voice data indicative of a surgical procedure performed on the patient (e.g., the patient 124). Further, and as indicated in block 322, the system 100 may obtain voice data indicative of a stage of a medical procedure being performed on the patient. For example, a caregiver 134, 136, 138 may state that anesthesia is being administered, that an initial incision is being made, that a closure process is being performed, etc. As indicated in block 324, the system 100 may obtain voice data indicative of a setting of a medical device used in a stage of a medical procedure performed on the patient. For example, the system 100 may obtain voice data indicative of a volumetric flow rate of anesthetic being administered to the patient, a voltage setting of an electrocauterization instrument, a position or intensity setting of a surgical light, a rotational speed of a drill, an inclination of a patient bed, etc.

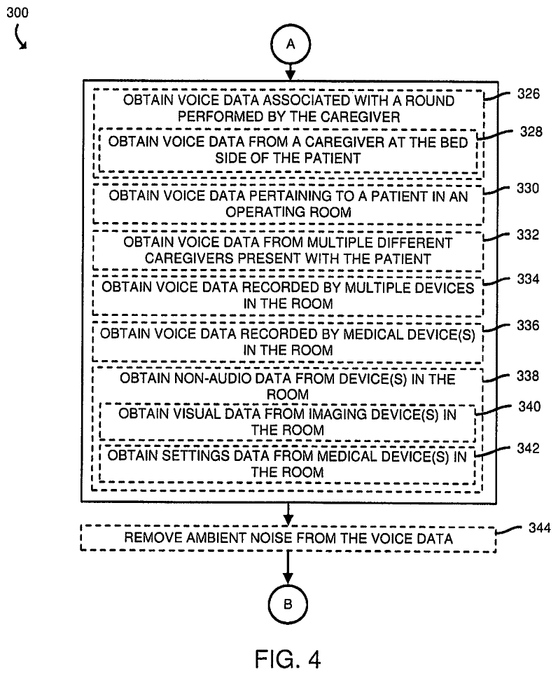

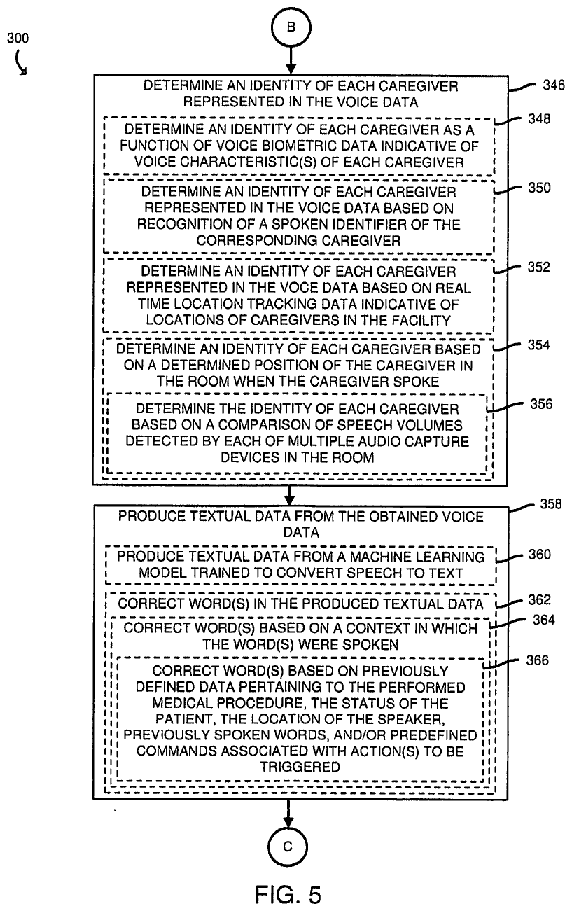

Referring now to FIG. 4, the system 100 may obtain voice data associated with a round performed by a caregiver (e.g., the caregiver 130), as indicated in block 326. For example, and as indicated in block 328, the system 100 may obtain voice data from a caregiver located at the bed side of a patient (e.g., the caregiver 130 at the bed side of the patient 120). As indicated in block 330, the system 100 may obtain voice data pertaining to a patient in an operating room (e.g., the patient 124 in the operating room 116). Additionally, and as indicated in block 332, the system 100 may obtain voice data from multiple different caregivers that are present with the patient (e.g., the caregivers 134, 136, 138 present with the patient 124). In some embodiments, the system 100 may obtain voice data recorded by multiple devices (e.g., multiple data capture devices 164) in the room (e.g., the room 116), as indicated in block 334. The system 100, in some embodiments, may also obtain voice data recorded by one or more medical device(s) in the room, as indicated in block 336. For example, a surgical light and/or a patient support apparatus (e.g., patient bed) may incorporate a data capture device 164 (e.g., a microphone) that may be used in the system 100 to obtain voice data. The system 100, in some embodiments, may also obtain non-audio data from one or more devices in the room, as indicated in block 338. As indicated in block 340, the system 100 may obtain visual data from one or more imaging devices (e.g., data capture devices 164, image capture devices 218) present in the room. For example, a surgical light may incorporate a camera which may capture one or more images of the surgical site for use by the system 100. As another example, a scope (e.g., a laparoscope, gastroscope, esophagoscope, etc.) operated by a caregiver may capture one or more images for use by the system 100. The system 100 may also obtain settings data from one or more medical devices (e.g., one or more medical devices 174) in the room (e.g., the room 116), such as a position or intensity of a surgical light, a voltage setting of an electrocauterization instrument, a rotational speed setting of a drill, an inclination of a patient bed, etc., as indicated in block 342. In some embodiments, the system 100 may remove ambient noise from the obtained voice data, such as by applying a bandpass filter, a dynamic noise reduction algorithm, and/or other noise reduction process to the obtained audio data, as indicated in block 344. Subsequently, the method 300 advances to block 346 of FIG. 5 in which the system 100 determines an identity of each caregiver represented in the voice data.

Referring now to FIG. 5, in determining an identity of each caregiver represented in the voice data, the system 100 may determine an identity of each caregiver as a function of voice biometric data indicative of one or more voice characteristics of each caregiver, as indicated in block 348. For example, the system 100 may compare dominant frequencies (e.g., formants) present in segments of obtained voice data to a biometric signature data set of dominant frequencies associated with each caregiver's voice and determine whether the formants present in each segment of the voice data satisfies a threshold similarity score to the biometric signature (e.g., from the data set) associated with one of the caregivers. If so, the system 100 identifies the corresponding segment of voice data as being spoken by the corresponding caregiver. Additionally or alternatively, the system 100 may determine an identity of one or more caregivers represented in the voice data based on recognition of a spoken identifier of the corresponding caregiver, as indicated in block 350. For example, a caregiver may speak his or her name or identification number in a sentence represented in the voice data. In other embodiments, a caregiver may speak another caregiver's name (e.g., in a conversation between multiple caregivers in a room) and the system 100 may utilize the spoken identification to narrow down the set of potential matches for other voices represented in the voice data collected from the same room (e.g., when comparing voice data to biometric signature data). Relatedly, and as indicated in block 352, the system 100 may determine an identity of each caregiver represented in the voice data based on real time location tracking data (e.g., obtained from the location tracking system 186) indicative of locations of caregivers in the facility 110. That is, if only one caregiver is determined to be present in the room in which the voice data is obtained, then the system 100 may associate the voice data with the one caregiver.

In other situations, in which multiple caregivers are detected in the room, the system 100 may limit the set of potential caregiver voice matches to those that are determined to be in the room (e.g., when performing a match based on voice biometric data). In some embodiments, the system 100 may determine an identity of a speaking caregiver represented in the voice data based on a determined position of each caregiver in the room when the caregiver spoke, as indicated in block 354. In doing so, and as indicated in block 356, the system 100 may determine the identity of each caregiver based on a comparison of speech volumes detected by each of multiple audio capture devices (e.g., data capture devices 164) in the room (e.g., the room 116). For example, if multiple microphones (e.g., data capture devices 164) are positioned at different locations in the room 116, the voice of a caregiver nearer to one of the microphones will be determined to be louder than the same voice detected by another microphone located farther away from the caregiver. As such, once a relative position of an identified caregiver in a room is determined (e.g., from voice biometric data and from a comparison of the volumes detected by multiple microphones at different locations in the room), the system 100 may ascribe, to the previously identified caregiver, other segments of voice data having similar differences in volume detected by the various microphones in the room.

Still referring to FIG. 5, the system 100, in the illustrative embodiment, produces textual data from the obtained voice data, as indicated in block 358. In doing so, and as indicated in block 360, the system 100 may produce textual data from a machine learning model (e.g., a neural network) trained to convert speech to text (e.g., trained using one or more reference sets of human-transcribed voice data). As indicated in block 362, the system 100 may correct one or more words in the produced textual data (e.g., by comparing the produced textual data to a dictionary of known words and replacing unidentified words in the textual data with the closest match in the dictionary). In some embodiments, in correcting one or more words, the system 100 may correct the words based further on a context in which the words were spoken (e.g., weighting possible matches to unidentified words in favor of known words that correspond with the context in which the unidentified words were spoken), as indicated in block 364. For example, and as indicated in block 366, the system 100 may correct one or more words based on previously defined data (e.g., from other portions of the textual data, from ADT data, from EMR data, and/or other sources) pertaining to the performed medical procedure, the status of the patient, the location of the speaker, previously spoken words, and/or predefined commands (e.g., trigger words, such as “begin recording notes for laparoscopy procedure”) associated with one or more actions to be triggered.

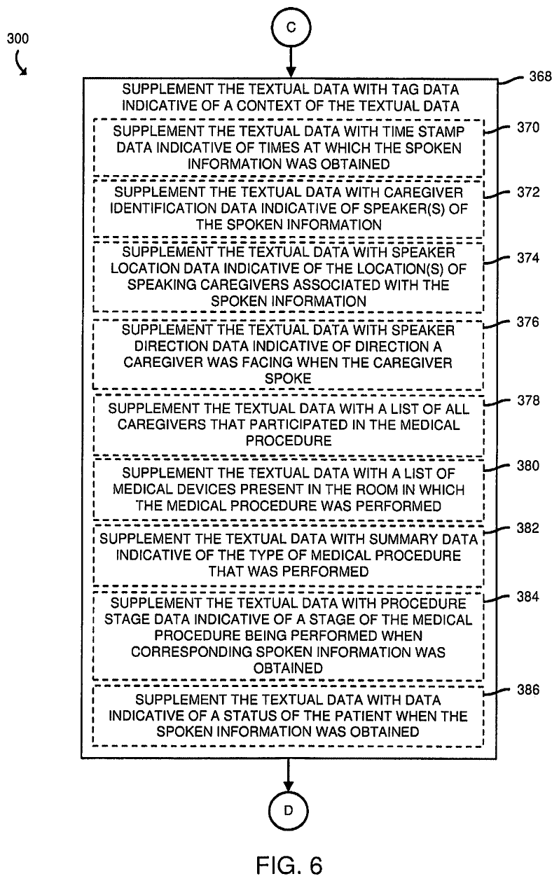

Continuing the method 300, and referring now to block 368 of FIG. 6, the system 100 may supplement the textual data with tag data (e.g., metadata), which may be embodied as any data indicative of the context of the textual data. The tag data may be generated from portions of the spoken information, data reported by devices in the room (e.g., medical devices), EMR data from the EMR system 182, location data from the location tracking system 186, and/or other sources of data obtained from any of the compute devices 140, 142, 150, 152, 154, 160, 162, 164, 170, 172, 174, 180, 182, 184, 186 of the system 100. In supplementing the textual data, and as indicated in block 370, the system 100 may supplement the textual data with time stamp data indicative of times at which the spoken information was obtained (e.g., a time associated with each spoken sentence). Additionally or alternatively, the system 100 may supplement the textual data with caregiver identification data, which may be embodied as any data that is indicative of the speaker(s) of the spoken information (e.g., caregiver name, identification number, etc. in association with each spoken sentence), as indicated in block 372. In some embodiments, the system 100 may supplement the textual data with speaker location data, which may be embodied as any data indicative of the location of a caregiver associated with the spoken information represented in the textual data (e.g., the location of the speaker of the spoken information), as indicated in block 374. The location may be expressed relative to a reference person (e.g., another caregiver), a reference object in the room (e.g., a patient bed), a coordinate system defined for the room, or any other coordinate system.

The system 100 may also supplement the textual data with speaker direction data, which may be embodied as any data indicative of a direction a speaker (e.g., caregiver) was facing when the caregiver spoke a portion of the spoken information represented in the textual data. The direction may be expressed relative another speaker, relative to one or more objects in the room, or relative to any other reference (e.g., geodetic north), as indicated in block 376. As indicated in block 378, the system 100 may supplement the textual data with a list of all caregivers that participated in a medical procedure to which the textual data pertains. Additionally or alternatively, the system 100 may supplement the textual data with a list of all medical devices present in the room in which the medical procedure was performed (e.g., from device identifiers reported by the medical devices themselves and/or based on spoken identifiers of the medical devices), as indicated in block 380. The system 100 may additionally or alternatively supplement the textual data with summary data indicative of the type of medical procedure that was performed, as indicated in block 382. Similarly, the system 100 may supplement the textual data with procedure stage data indicative of a stage of the medical procedure being performed when corresponding spoken information (e.g., represented by the textual data) was spoken, as indicated in block 384. The system 100 may also supplement the textual data with data indicative of a status of the patient when the spoken information was obtained, as indicated in block 386.

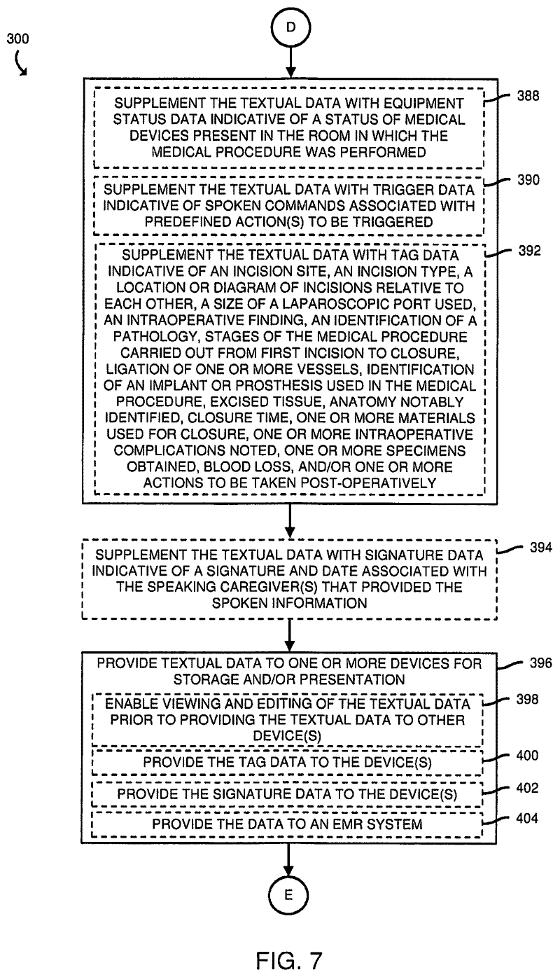

Referring now to FIG. 7, the system 100 may supplement the textual data with equipment status data which may be embodied as any data indicative of a status of one or more medical devices (e.g., the other devices 174 in FIG. 1) present in the room (e.g., the room 116) in which the medical procedure was performed, as indicated in block 388. In doing so, the system 100 may utilize data reported directly from the medical devices, from spoken information (e.g., from one or more of the caregivers 134, 136, 138) regarding the medical devices, and/or other sources. As indicated in block 390, the system 100 may supplement the textual data with trigger data which may be embodied as any data indicative of one or more spoken commands associated with one or more predefined actions to be taken (e.g., performed by the system 100), such as to begin producing documentation (e.g., from spoken information) for a particular medical procedure, to begin producing documentation (e.g., from spoken information) about a visit to a patient's room during a hospital round, or to conclude the production of documentation regarding a medical procedure. The trigger data may indicate, for example, the command that was spoken, the time the command was spoken, and the action that was performed in response. As indicated in block 392, the system 100 may supplement that textual data with tag data indicative of any of an incision site, an incision type, a location or diagram (e.g., obtained from a data capture device 164, image capture device 218, or other source) of incisions relative to each other, a size of a laparoscopic port used, an intraoperative finding, an identification of a pathology, stage(s) of a medical procedure carried out from first incision to closure, ligation of one or more vessels, identification of an implant or prosthesis used in the medical procedure, excised tissue, anatomy notably identified, closure time, one or more materials used for closure, one or more intraoperative complications noted, one or more specimens obtained, a quantity of blood loss, and/or one or more actions to be taken post-operatively.

In some embodiments, as indicated in block 394, the system 100 may supplement the textual data with signature data, which may be embodied as any data indicative of a signature and date associated with the speaking caregiver(s) that provided the spoken information represented in the textual data. For example, the system 100 may add, to the textual data, the date that the spoken information was obtained (e.g., spoken by a corresponding caregiver and detected by the system 100) and a stored image of a handwritten signature of each corresponding caregiver. As indicated in block 396, the system 100 may provide the textual data to one or more devices for storage and/or presentation. In doing so, and as indicated in block 398, the system 100 may enable viewing and editing of the textual data prior to providing the textual data to other devices. For example, the system 100 may present the textual data to the caregiver who initially provided the spoken information (e.g., in the audio data) via the caregiver's mobile compute device (e.g., mobile compute device 140, 142) or a nearby compute device (e.g., a presentation device 150, 152, 154) for review, editing, and confirmation of accuracy by the corresponding caregiver(s) prior to providing the textual data to other devices in the system 100. As indicated in block 400, in providing the textual data to one or more devices, the system 100 may additionally provide the tag data, discussed above, to the one or more devices for storage and/or presentation. Additionally or alternatively, and as indicated in block 402, the system 100 may provide the signature data, discussed above in block 394, to the one or more devices. The system 100 may provide the data (e.g., the textual data, the tag data, the signature data) to an electronic medical records system (e.g., the EMR system 182), as indicated in block 404.

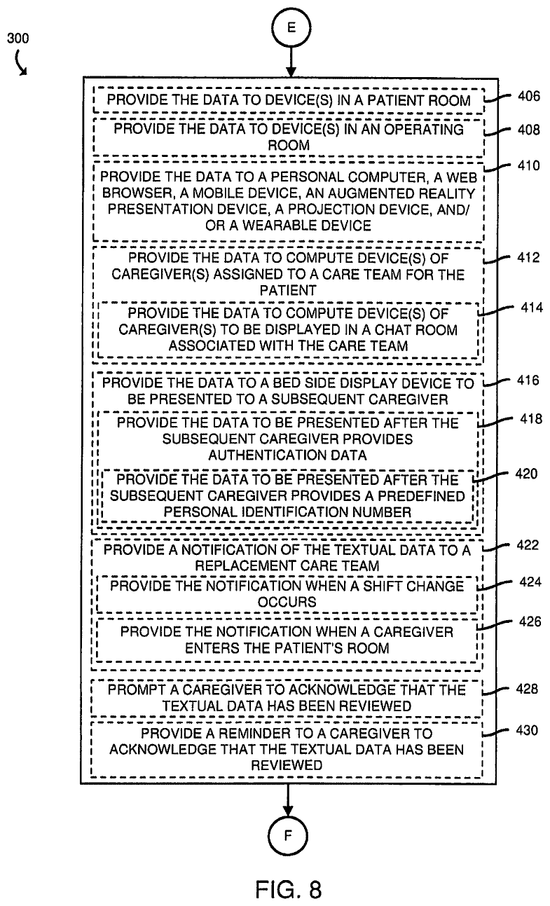

Referring now to FIG. 8, the system 100 may provide the data to one or more devices (e.g., presentation device(s) 150, 152) in a patient room (e.g., patient rooms 112, 114), as indicated in block 406. Similarly, the system 100 may provide the data to one or more devices in an operating room (e.g., the presentation device(s) 154 in the operating room 116), as indicated in block 408. As indicated in block 410, the system 100 may provide the data to a personal computer, a web browser (e.g., as a web page, rather than in the user interface of a native application, executed on a compute device), a mobile device (e.g., a mobile compute device 140, 142), an augmented reality presentation device (e.g., eyewear worn by a caregiver or a projection device that overlays visual information onto other visual information from the environment), or other device, as indicated in block 410. As indicated in block 412, the system 100 may provide the data to one or more compute devices of caregiver(s) assigned to a care team for a patient to whom the data relates. In doing so, and as indicated in block 414, the system 100 may provide the data to compute device(s) of caregiver(s) to be displayed in a chat room (e.g., a user interface configured to present messages communicated between multiple participants, such as in chronological order) associated with the care team. In such embodiments, the patient care coordination system 180 may determine the identities of the caregivers associated with a care team for a corresponding patient and send the data to the mobile compute device(s) associated with those caregivers.

In some embodiments, the system 100 may provide the data to a bed side display device (e.g., a presentation device 150, 152) to be presented to a subsequent caregiver (e.g., a caregiver assigned to the next shift), as indicated in block 416. In doing so, the system 100 may, in some embodiments, provide the data after the subsequent caregiver provides authentication data (e.g., proving the identity of the subsequent caregiver), as indicated in block 418. For example, and as indicated in block 420, the system 100 may provide the data after the subsequent caregiver provides a predefined personal identification number (PIN) verifying the identity of the subsequent caregiver.

Relatedly, the system 100 may provide a notification of the textual data to a replacement care team assigned to the corresponding patient, as indicated in block 422. In doing so, the system 100 may provide the notification when a shift change occurs, as indicated in block 424. As indicated in block 426, the system 100 may provide the notification when a caregiver (e.g., the subsequent caregiver) enters the room of the patient (e.g., as detected by the location tracking system 186). In some embodiments, the system 100 may prompt (e.g., through the caregiver's mobile compute device 140, 142, a presentation device 150, 152, 154, etc.) a caregiver (e.g., the caregiver notified of the existence of the textual data) to acknowledge that the textual data (and any associated data, such as tag data) has been reviewed by the caregiver, as indicated in block 428. Relatedly, the system 100 may provide a reminder (e.g., through the caregiver's mobile compute device 140, 142, a presentation device 150, 152, 154, etc.) to a caregiver to acknowledge that the textual data has been reviewed (e.g., after a predefined amount of time has elapsed since the notification was provided to the caregiver, prior to the performance of a scheduled medical procedure on the patient, etc.), as indicated in block 430. In the illustrative embodiment, the method 300 loops back to block 304 of FIG. 3 to continue to obtain voice data pertaining to patient(s) and to perform the other operations associated with the method 300 described above.

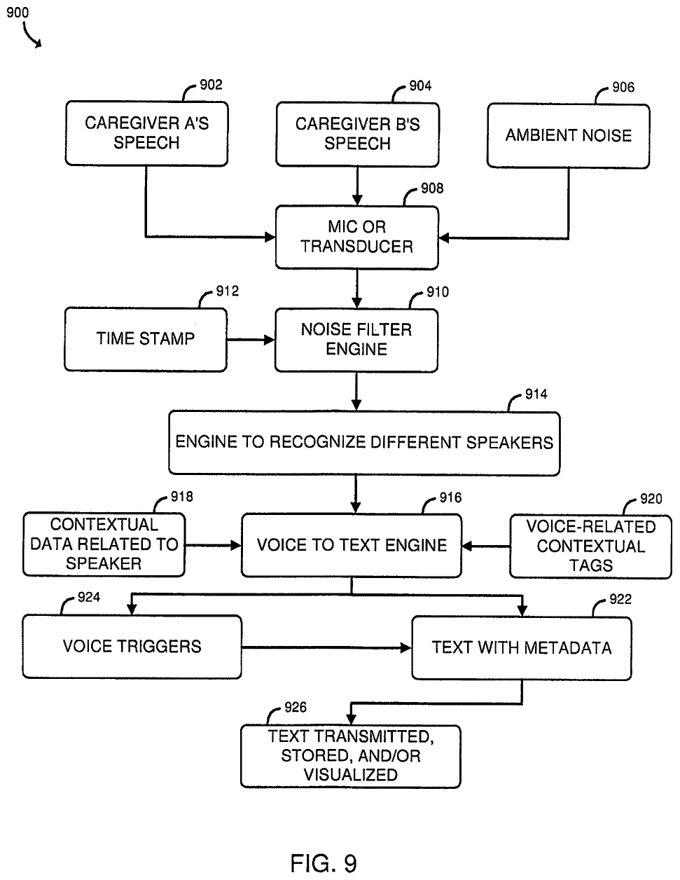

Referring now to FIG. 9, an illustrative embodiment of a flow 900 of data through the system 100 during the execution of the method 300 is shown. As shown, a caregiver's speech (e.g. spoken words), as represented in block 902 is obtained by a microphone or transducer 908 (e.g., a data capture device 160, 162, 164, an audio capture device 214). The microphone or transducer 908, in the illustrative embodiment, also obtains the speech of another caregiver (e.g., in the same room), as represented by block 904. Further, the microphone or transducer 908, in the illustrative embodiment, obtains ambient noise (e.g., background noise in the room), as indicated in block 906. Together, the obtained speech and ambient noise from blocks 902, 904, 906 constitutes audio data. The microphone or transducer 908 provides the audio data to a noise filter engine, represented by block 910. The noise filter engine, represented by block 910, which may be embodied as a noise reduction algorithm executed by corresponding hardware (e.g., a processor executing instructions, reconfigurable circuitry, application specific circuitry, etc.) in any of the devices of the system 100, reduces the presence of the ambient noise (e.g. from block 906) in the audio data. Additionally, the system 100 may associate time stamp data (e.g., any data indicative of a time when the audio data was obtained) with the audio data, as represented by block 912.

Further, the system 100 provides the audio data combined with the time stamp data, to an engine to recognize different speakers, as indicated in block 914. The engine to recognize different speakers may be embodied as an algorithm to identify speakers based on voice biometric data (e.g., dominant frequencies known as formants), executed by corresponding hardware (e.g., a processor executing instructions, reconfigurable circuitry, application specific circuitry, etc.) in any of the devices of the system 100. Further, and as indicated in block 916, a voice to text engine (e.g., a voice to text algorithm executed by any device of the system 100) obtains the audio data and produces textual data from the audio data. In doing so, the system 100 may utilize contextual data, represented by block 918, relating to the speaker(s) associated with the audio data. The contextual data (e.g., corresponding to block 368 of FIG. 6 and sub-blocks thereof) may include, for example, words previously spoken, the location of the speaker (e.g., relative to a patient), identities of caregivers (e.g., caregivers present in a room in which the audio data was obtained), equipment in the room, and/or the direction one or more speakers are facing.

Additionally or alternatively, the voice to text engine represented by block 916 may utilize voice-related contextual tags (e.g., the tag data described with reference to block 392 of FIG. 7), as indicated in block 920. As indicated in block 922, the system 100 produces text (e.g., textual data) with metadata (e.g., tag data). In doing so, the system 100 may additionally identify voice triggers that were spoken by one or more caregivers and tag the voice triggers (e.g., in a process similar to that described with reference to block 390 of FIG. 7) in the metadata (e.g., tag data). Further, the system 100 provides (e.g., transmits) the data (e.g., textual data, metadata including tag data, etc.) to one or more devices, stores the data (e.g., in the EMR system 182 and/or other devices of the system 100), and/or visualizes (e.g., presents on a display device) the data (e.g., on a presentation device 150, 152, 154, a mobile compute device 140, 142, etc.).

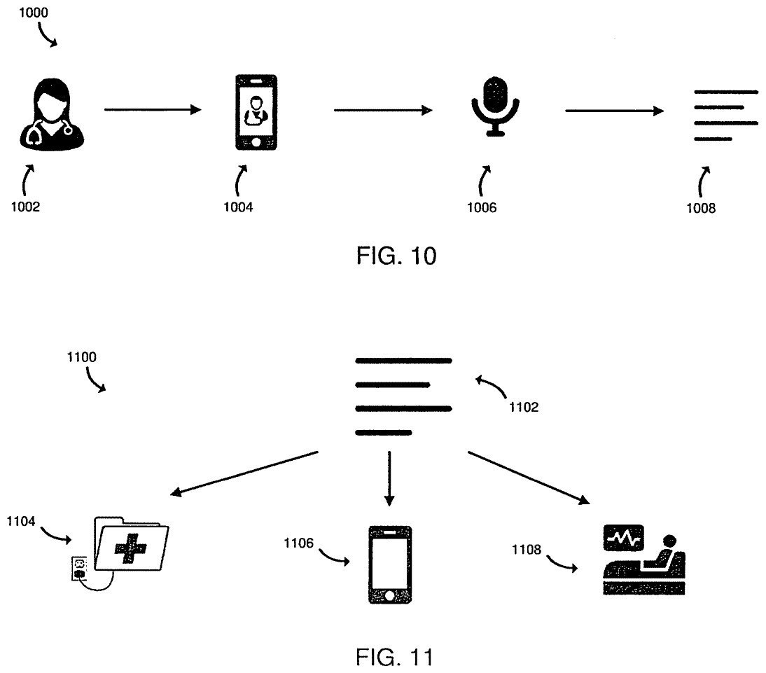

Referring now to FIG. 10, in an example workflow 1000 for the system 100, a caregiver 1002 may enter a room (e.g., the room 112 of the patient 120). In response, the system 100 detects that the caregiver 1002 has entered the room (e.g., using the location tracking system 186). In a subsequent step, the caregiver 1002 opens an application on the mobile compute device 1004, which is similar to the mobile compute device 140. The mobile application, in the illustrative embodiment, is designed to communicate with the patient care coordination system 180. For example the mobile application may be embodied as a mobile application associated with the Voalte® Platform from Hill-Rom Services, Inc.

The mobile compute device 1004, in the illustrative embodiment, receives a notification from the location tracking system 186 or the patient care coordination system 180 (e.g., the Voalte® Platform) that the caregiver 1002 has entered the patient's room, and in response, displays in the mobile application, information (e.g., data provided from the EMR system 182) pertaining to the patient in the room (e.g., the patient 120 in the room 112). In step 1006, the caregiver begins to report (e.g., verbally) on the patient and the system 100 (e.g., the mobile compute device 1004, which is similar to the mobile compute device 140) captures notes (e.g., the audio data, to be converted to textual data) via a microphone (e.g., the audio capture device 214). Subsequently, in step 1008, the system 100 (e.g., the mobile compute device 1004, 140, or another device in the system 100 that receives the audio data from the mobile compute device 1004, 140) converts the notes (e.g., the audio data) to textual data. Referring briefly to FIG. 11, in a subsequent process 1100, a set of textual data 1102 (similar to the textual data associated with step 1008 in FIG. 10) may be disseminated to one or more devices of the system 100, such as an EMR system 1104 (similar to the EMR system 182), a mobile compute device 1106 (e.g., of another caregiver, such as the mobile compute device 142 of the caregiver 132), and/or a display device in a Opatient room (e.g., the presentation device 150 in the patient room 112).

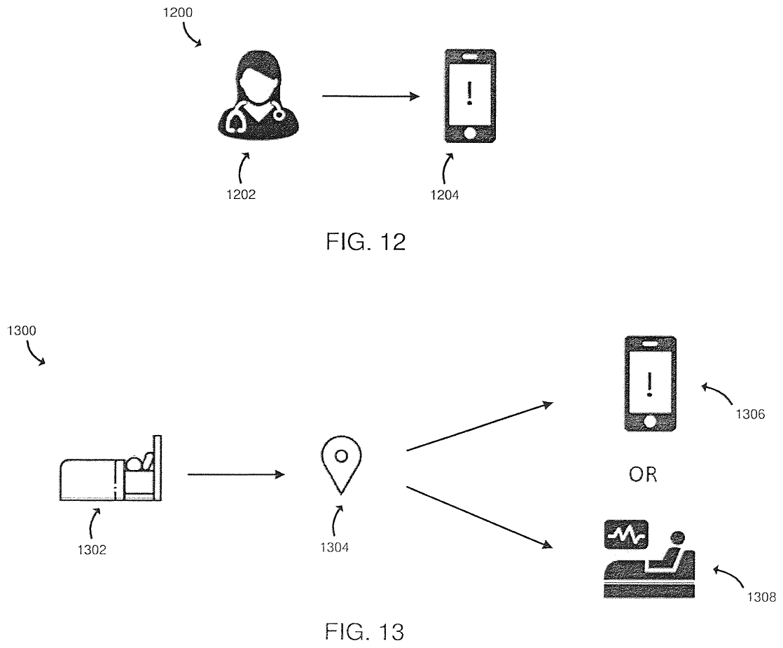

Referring now to FIG. 12, a flow 1200 for providing smart notifications for rounding notes (e.g., textual data produced, by the system 100, from audio data) is shown. In a first step 1202, a caregiver signs into a mobile application associated with the patient care coordination system 180 (e.g., the Voate® Platform from Hill-Rom Services, Inc.). In a subsequent step 1204, the mobile application (e.g., the mobile compute device executing the mobile application), presents a notification that there are unread rounding notes (e.g., textual data produced, by the system 100, from audio data) from the previous shift. Referring now to FIG. 13, in another flow 1300, a caregiver enters a room of a patient, as indicated in step 1302. Subsequently, in step 1304, a real time location system (e.g., the location tracking system 186) associates the caregiver's location with a particular patient's room. Afterwards, the caregiver may receive a notification that there are unread rounding notes (e.g., in step 1306) and/or a bedside display may notify the caregiver of unread rounding notes, as indicated in block 1308.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

1. A patient care coordination system comprising:
a cloud based data center configured to enable communication among caregivers at a healthcare facility and receive information from devices at the healthcare facility;
an electronic medical records (EMR) system configured to obtain electronic medical record data pertaining to patients, store the electronic medical record data, and provide the electronic medical record data upon request;
an admission, discharge, transfer (ADT) system configured to store data indicative of patients that have been admitted to the healthcare facility, including when the patients were admitted, unique identifiers associated with the patients, and references to the medical record data;
a location tracking system configured to track the locations of caregivers throughout the healthcare facility based on detections of tracking devices moving with the caregivers; and
a mobile compute device configured to be carried by or attached to a caregiver, the mobile compute device being configured to execute instructions which process audio signals originating as voice inputs spoken by the caregiver, filter the audio signals, and convert the audio signals into textual data;
wherein at least one or more of the EMR system, ADT system, location tracking system, and mobile compute device are configured to execute further instructions to supplement the textual data with location data of the caregiver received from the location tracking system, determine that a patient is associated with the caregiver based on association data received from the location tracking system and the ADT system, obtain at least some of the electronic medical record data regarding the patient from the EMR system and provide the at least some of the medical record data to the caregiver, determine medical notes regarding the patient included in the textual data, and record the medical notes in the EMR system in a corresponding record for the patient.

2. The patient care coordination system of claim 1, wherein a first tracking device of the tracking devices is combined with the mobile compute device.

3. The patient care coordination system of claim 1, wherein the medical notes comprise rounding notes regarding a status of the patient observed by the caregiver during a visit to the patient's room during a round.

4. The patient care coordination system of claim 1, wherein the medical notes comprise procedure notes regarding a medical procedure performed on the patient.

5. The patient care coordination system of claim 4, wherein the medical procedure to which the procedure notes pertain includes a procedure performed during surgery.

6. The patient care coordination system of claim 1, wherein the mobile compute device is further configured to display unread rounding notes that were converted to rounding textual data from speech signals originating from one or more other caregivers during a previous shift.

7. The patient care coordination system of claim 6, wherein the unread rounding notes pertain to a particular patient and are displayed on the mobile compute device in response to the location system detecting that the caregiver has entered a corresponding room of the particular patient.

8. The patient care coordination system of claim 6, wherein the mobile compute device is configured to notify the caregiver of unread rounding notes and the unread rounding notes are displayed on the mobile compute device in response to a manual input by the caregiver on the mobile compute device after being notified.

9. The patient care coordination system of claim 1, further comprising a bedside display device situated in a patient room assigned to the patient and wherein the bedside display device is configured to display unread rounding notes that were converted to rounding textual data from speech signals originating from one or more other caregivers during a previous shift.

10. The patient care coordination system of claim 7, wherein the unread rounding notes are displayed on the bedside display device in response to the location system detecting that the caregiver has entered a corresponding room of the respective patient.

11. The patient care coordination system of claim 1, wherein the mobile compute device is further configured to produce the textual data using a machine learning model trained to convert speech to text.

12. The patient care coordination system of claim 1, wherein the mobile compute device is further configured to use voice recognition to identify other people at the caregiver's location.

13. The patient care coordination system of claim 1, further comprising a second mobile compute device of a second caregiver, wherein the location tracking system is further configured to determine that the second caregiver has entered a room associated with the patient, and wherein the second mobile compute device is configured to receive a notification of the textual data entered by the caregiver using the first mobile compute device.

14. The patient care coordination system of claim 13, wherein the second mobile compute device is configured to prompt the second caregiver to acknowledge that the textual data has been reviewed.

15. The patient care coordination system of claim 1, further comprising a bedside display device situated in a patient room assigned to the patient and wherein the bedside display device is configured to display at least some of the textual data entered by the caregiver using the mobile compute device.

16. The patient care coordination system of claim 15, wherein the textual data is displayed on the bedside display device in response to the location system detecting that a second caregiver has entered the patient room.

17. The patient care coordination system of claim 1, wherein the mobile compute device is configured to display the textual data to the caregiver for review and editing before the textual data is provided to another device for storage or presentation.

18. The patient care coordination system of claim 1, wherein the mobile compute device is further configured to supplement the textual data with data indicative of a medical procedure being performed when the one or more voice inputs were spoken, a status of the patient when the one or more voice inputs were spoken, voice inputs that were previously spoken by the caregiver, or one or more predefined words associated with predefined commands of the voice inputs.

19. The patient care coordination system of claim 1, wherein the mobile compute device is further configured to supplement the textual data with data indicative of one or more of the following: a listing of equipment located in an operating room in which a medical procedure is being performed on the patient, a type of medical procedure being performed on the patient, procedure stage data indicative of a present stage of the medical procedure performed on the patient when the voice inputs are being spoken, patient status data indicative of a status of the patient when the voice inputs are being spoken, equipment status data indicative of a status of equipment present in a room in which the medical procedure is being performed, and signature data indicative of a signature and date associated with the caregiver who spoke the voice inputs represented in the textual data.

20. The patient care coordination system of claim 19, wherein the mobile compute device is further configured to supplement the textual data with data indicative of one or more of the following: an incision site, an incision type, a location or diagram of incisions relative to each other, a size of a laparoscopic port used, an intra-operative finding, an identification of a pathology, stages of the medical procedure carried out from first incision to closure, ligation of one or more vessels, identification of an implant or prosthesis used in the medical procedure, excised tissue, anatomy notably identified, closure time, one or more materials used for closure, one or more intraoperative complications noted, one or more specimens obtained, blood loss, or one or more actions to be taken post-operatively.
